(19) United States
(12) Patent Application Publication
(10) Pub. No.: US 2014/0183030 A1
(43) Pub. Date: Jul. 3, 2014

(54) PROCESS FOR THE PURIFICATION OF A CRUDE SOLVENT STREAM COMPRISING AN N-ALKYLPYRROLIDONE

(71) Applicant: BASF SE, Ludwigshafen (DE)

(72) Inventors: Christoph Sigwart, Jeollanam-do (KR); Jörg Erbes, Karlsruhe (DE); Martin Weber, Maikammer (DE); Gerhard Lange, Schriesheim (DE); Jutta Vonend, Bad Durkheim (DE); Cornelis Hendricus De Ruiter, Mannheim (DE)

(73) Assignee: BASE SE, Ludwigshafen (DE)

(21) Appl. No.: 14/140,861

(22) Filed: Dec. 26, 2013

Related U.S. Application Data
(60) Provisional application No. 61/746,583, filed on Dec. 28, 2012.

(51) Int. Cl. C07D 207/06

(52) U.S. Cl. CPC C07D 207/06
USPC 203/37

(57) ABSTRACT

N-alkylpyrrolidone and, as contaminant alongside this, the corresponding N-alkyl-succinimide, to give a pure solvent stream which meets the requirements for use in a process for the production of polymers, where compounds of higher and lower boiling point than the N-alkylpyrrolidone are removed by distillation, which comprises, prior to, during, or after the distillative purification process, adding a hydroxide of an alkali metal or of an alkaline earth metal in a molar amount corresponding to a molar ratio of at least 0.1:1 for this hydroxide with respect to the respective N-alkylsuccinimide in the appropriate solvent stream present prior to, during, or after the distillative purification process.

The invention relates to a process for the purification of a crude solvent stream comprising an N-alkylpyrrolidone and, as contaminant alongside this, the corresponding N-alkylsuccinimide, to give a pure solvent stream which meets the requirements for use in a process for the production of polymers. Polymers are frequently produced via polymerization processes or polycondensation processes in the presence of a polar aprotic solvent, those used being N-alkyl-2-pyrrolidones, hereinafter abbreviated to NAP. A particularly suitable NAP is N-methyl- or M-ethylpyrrolidone, in particular N-methylpyrrolidone, hereinafter abbreviated to NMP.

Contaminated solvent arises in the above processes and for economic and environmental reasons requires treatment and recycling into the process.

The solvent used in the above processes must therefore comply with the criteria for what is known as pure NAP, therefore having at least 99.0% by weight NAP content or else at least 99.5% by weight NAP content, or else at least 99.8% by weight NAP content, based in each case on the total weight of the pure NAP stream, and at most the following contents of components detrimental to specification: at most 0.1% by weight of water and at most 0.02% by weight of N-alkylsuccinimide, or else at most 0.01% by weight of N-alkylsuccinimide, hereinafter abbreviated to NAS, based in each case on the total weight of the pure NAP stream.

Higher NAS contents in the NAP solvent have a disadvantageous effect on the color of the polymers. This is surprising because not only NAP itself but also NAS, which can be produced by way of example via oxidation of NAP by atmospheric oxygen, are colorless substances. However, for the reasons described the market demands polymers with minimized intrinsic color.

Current thinking is that there is a causal connection between the NAS produced via oxidation of the NAP, for example the N-methylsuccinimide (NMS) produced via oxidation of N-methylpyrrolidone (NMP):

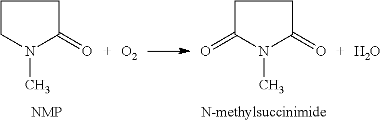

and the undesired intrinsic color of the final polymer product.

It is believed that NAS is a precursor for higher-molecular-weight colorant components which impair the intrinsic color of the polymers.

Prior to recycling into the production process for the polymers, NAP-containing recycling steams are therefore purified via final distillation in a traditional distillation column sufficiently to give a pure NMP which complies with the criteria defined above.

However, the removal of the N-alkylsuccinimides from the corresponding N-alkyl-pyrrolidones to give the purity demanded is a difficult problem in distillative separation technology, because the materials have quite similar boiling points: by way of example, the boiling point of NMP at atmospheric pressure is about 204° C., and the boiling point of NMS at atmospheric pressure is about 238° C. If conventional distillative separation were used for this purpose it would incur high energy cost.

It was therefore an object of the invention to provide a process for the purification of a crude solvent stream comprising an N-alkylpyrrolidone and, as contaminant alongside this, the corresponding N-alkylsuccinimide, to give a pure solvent steam which complies with the requirements for use in a process for the production of polymers, which can be carried out in a manner that is technically simple and without high apparatus cost, and which reliably leads to the purity demanded from the pure solvent stream.

The object is achieved via a process for the purification of a crude solvent stream comprising an N-alkylpyrrolidone and, as contaminant alongside this, the corresponding N-alkylsuccinimide, to give a pure solvent stream which meets the requirements for use in a process for the production of polymers, where compounds of higher and lower boiling point than the N-alkylpyrrolidone are removed by distillation, which comprises, prior to, during, or after the distillative purification, adding a hydroxide of an alkali metal or of an alkaline earth metal in a molar amount corresponding to a molar ratio of at least 0.1:1 for this hydroxide with respect to the respective N-alkylsuccinimide in the appropriate solvent stream present prior to, during, or after the distillative purification.

It has been found that the above technical object can be achieved in a simple and reliable manner via the specific selection above of the molar ratio, based on the content of the appropriate N-methylsuccinimide, in which the hydroxide is added to the solvent stream.

The concentration of NMS in the solvent steam is preferably determined by gas chromatography.

The expressions molar amount and molar ratio usually have the following meaning, as in Wikipedia:

Molar amount is the term used for the quantity of substances, in particular in stoichiometry. Said molar amount has been arbitrarily selected as an agreed base unit in the International System of Units (SI). The unit of molar amount is the mol, an SI base unit.

Use of the term mol requires precise definition of the underlying number of particles (in particular atoms and molecules); one mol of a substance comprises, subject to current limits of precision of measurement, about 6.02214129(27) 10²³ of such particles (Avogadro number N_{A}). These particles can also be imaginary fragments of actual particles (e.g. ¼ of a molecule or ion)—the term used here being equivalent particles, sometimes abbreviated to equivalents—and the outmoded unit eq for equivalent weights can therefore be replaced by mol without any changes to well-established numeric values. Many details in this connection were found in the German standard DIN 32625. However, this was withdrawn in April 2006, because it was no longer required.

The following relationship applies to the molar amount nx and the mass mx of a quantity of a pure substance X and the molar mass Mx thereof:

nₓ=mₓ/Mₓ

The expression molar ratio is correspondingly the term used for the ratio of two molar amounts.

The process for the production of polymers is preferably a process for the production of one or more polyarylene ether sulfones.

Polyarylene ether sulfones are known by way of example with trademark Ultrason® from BASF SE, and comprise in particular polyether sulfones (Ultrason® E), polysulfones (Ultrason® S), and polyphenyl sulfones (Ultrason® P).

Ultrason® E, Ultrason® S, and Ultrason® P are transparent plastics with high temperature resistance. They are used in many applications in engineering and in the electrical/electronics sector. There are also numerous reasons for a use as replacement for glass, metal, ceramic, and porcelain in the food-and-drinks sector and household sector: heat resistance extending to 180° C. or short periods at 220° C., good mechanical properties and high breakage resistance, resistance to superheated steam, and excellent resistance to chemicals.

Ultrason® E, S, and P are amorphous thermoplastic polymers with the following underlying structure:

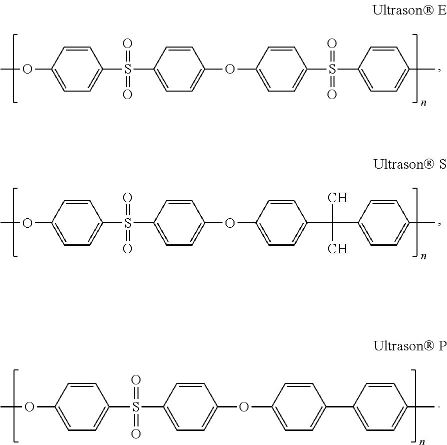

Moldings made of Ultrason® not only have high dimensional stability but also strength, stiffness, and toughness, these properties extending to the vicinity of the glass transition temperature.

The most important features of Ultrason® are:
properties independent of temperature
very high long-term service temperatures
good dimensional stability
high stiffness
high mechanical strength
good electrical insulation capability
advantageous dielectric properties
very advantageous fire performance
exceptional resistance to hydrolysis

The three Ultrason® parent polymers are amorphous thermoplastics and are transparent. However, by virtue of the high temperatures required during their production and processing they have a certain intrinsic color (pale golden yellow to ocher) which prevents achievement of the theoretically possible transmittance values for visible light. The qualities achievable currently are nevertheless suitable for very many transparent applications. Ultrason® also has high refractive indices in the visible wavelength region, and it therefore has another use in functional optical applications, for example lenses for electronic cameras.

Polyarylene ether sulfones are frequently produced via polycondensation in the presence of, as polar aprotic solvent, an N-alkyl-2-pyrrolidone, hereinafter abbreviated to NAP. N-Methyl- or N-ethylpyrrolidone are particular N-alkyl-2-pyrrolidones that are used, and in particular N-methylpyrrolidone is used. Processes of this type are disclosed by way of example in U.S. Pat. No. 4,870,153, EP-A 113 112, EP-A 297 363, and EP-A 135 130.

The process for the production of the polyarylene ether sulfones is in particular a polycondensation of aromatic bishalogen compounds and aromatic bisphenols or salts thereof in the presence of at least one alkali metal carbonate or ammonium carbonate.

It is preferable that the hydroxide of an alkali metal or of an alkaline earth metal is added in a molar amount corresponding to a molar ratio of from 0.1:1 to 10:1 with respect to the respective N-alkylsuccinimide in the appropriate solvent stream present prior to, during, or after the distillative purification.

It is further preferable that the hydroxide of an alkali metal or of an alkaline earth metal is added in a molar amount corresponding to a molar ratio of from 0.2:1 to 2:1 with respect to the respective N-alkylsuccinimide in the appropriate solvent stream present prior to, during, or after the distillative purification.

The N-alkylpyrrolidone is preferably N-ethyl- or N-methylpyrrolidone, in particular N-methylpyrrolidone.

In particular, the crude solvent stream is a recycling steam from a plant for the production of polyarylene ether sulfones via polycondensation of aromatic bishalogen compounds and of aromatic bisphenols or salts thereof in the presence of at least one alkali metal carbonate or ammonium carbonate or alkali metal hydrogencarbonate or ammonium hydrogencarbonate in N-methylpyrrolidone as solvent, comprising from 60 to 90% by weight of water, from 10 to 40% by weight of N-methylpyrrolidone, and as contaminant detrimental to specification, up to 5000 ppm by weight of N-methyl-succinimide and, alongside this, up to 1000 ppm by weight of other substances with higher boiling point than N-methylpyrrolidone, in particular inorganic salts, based in each case on the total weight of the recycling stream, where the entirety of the components gives 100% by weight,

to give a pure N-methylpyrrolidone steam which can be returned to the plant for the production of polyarylene ether sulfones.

It is preferable that the purification is carried out via final distillation in a final column, upstream of which there is a preliminary purification via evaporation in one or more evaporator stages to reduce the content of other substances with higher boiling point than N-methylpyrrolidone, in particular inorganic salts, where the final column receives one or more feed streams, and where the bottom stream from the final evaporator stage is discharged, and the bottom steam from the final column is entirely recycled into the final evaporator stage.

The preliminary purification via evaporation is preferably carried out in two or three evaporator stages.

The addition of the hydroxide of the alkali metal or of the alkaline earth metal advantageously takes place in the first evaporator stage, where the hydroxide of the alkali metal or of the alkaline earth metal is introduced together with the recycling stream or separately therefrom into the first evaporator stage.

In addition, or as an alternative, the addition of the hydroxide of the alkali metal or of the alkaline earth metal can take place in the second evaporator stage.

In another embodiment, the addition of the hydroxide of the alkali metal or of the alkaline earth metal can take place in the third evaporator stage.

In another embodiment, the addition of the hydroxide of the alkali metal or of the alkaline earth metal can take place after the distillation.

The hydroxide of the alkali metal or of the alkaline earth metal can be added in the solid state, or preferably in the form of solution in water, where the concentration of the hydroxide of the alkali metal or of the alkaline earth metal is preferably from 1 to 50% by weight, more preferably from 2 to 25% by weight, in particular from 5 to 10% by weight, based in each case on the total weight of the solution.

It is preferable that the hydroxide of the alkali metal or of the alkaline earth metal is sodium hydroxide.

It is further preferable that the hydroxide of the alkali metal or of the alkaline earth metal is potassium hydroxide.

The distillative purification can be carried out continuously or batchwise, preferably continuously.

The invention is explained in more detail below with reference to inventive examples:

Syntheses of polyaryl ether sulfones were carried out in NaOH-free NMP (comparative example) and in NMP to which NaOH had been added (inventive examples).

Analytical Determinations
1) Intrinsic viscosity (IV) of the polyaryl ether sulfones:

The intrinsic viscosity (IV) of the polyaryl ether sulfones was determined in 1% N-methylpyrrolidone solution at 25° C. (DIN EN ISO 1628-1).
2) N-Methylsuccinimide content of the N-methylpyrrolidone used:

The N-methylsuccinimide content of the N-methylpyrrolidone used was determined by gas chromatography.
3) Color determination on the polyaryl ether sulfone:

The optical properties of the granulated polyaryl ether sulfone material were determined via color measurements on polymer plaques produced via injection molding from the granulated polyaryl ether sulfone material. Color was determined on the polyaryl ether sulfone plaques in comparison with a measurement on a reference specimen. The reference specimen features a particularly pale and colorless appearance. The color of the product is described within the L*a*b* color space (a three-dimensional system of coordinates), the b* and L* values in said system being relevant for the specimens under consideration. The b* axis describes the blue or yellow component of a color, negative values representing blue and positive values representing yellow. The L* axis describes the lightness of the color.

The b* and L* values for the specimens are compared with the b* and L* values for the reference specimen and are stated in the form of difference from the reference specimen:

The greater the dL* value, where dL*=L*_{specimen}−L*_{reference}, the lighter is the color of the product.

The smaller the db* value, where db*=b*_{specimen}−b*_{reference}, the “bluer” is the appearance of the product, with therefore less yellow color tinge.

The measurement equipment used to carry out the color measurement was a Datacolor DC 3890.

### COMPARATIVE EXAMPLE

The polyaryl ether sulfone synthesis used freshly distilled NMP to which no NaOH was added. The NMP comprised 62 ppm of N-methylsuccinimide.

The monomers, dichlorodiphenyl sulfone (574 g), and dihydroxydiphenyl sulfone (500 g) were dissolved in NMP (1050 ml). K₂CO₃ (290 g) was added, and the reaction medium was heated at 190° C. for 4 h, with supply of nitrogen at 30 l/h, and stirring. Further NMP (1.950 ml) was then added, and 15 l of methyl chloride gas were introduced at temperatures of from 120° C. to 150° C. over a period of 1 h. The suspension was then cooled below 80° C., and then filtered. The polymer was precipitated in water, and the precipitated polymer particles were washed with boiling water, and dried at 150° C. in vacuo for 20 h.

The IV of the resultant polymer was 56.5 ml/g.

The polymer was then processed at 360° C. in a twin-screw extruder (PTW16) to give granules, and injection-molded in the form of plaques. The optical properties of the plaques were measured.

Color result: dL*=−8.40; db*=6.37

### Example 1 of the Invention

The synthesis used the same freshly distilled NMP from the comparative experiment, but 0.2 g of a 2 molar aqueous NaOH solution had also been added to each liter of this NMP. The molar NaOH/NMS ratio was 0.7.

The polymer synthesis and polymer work-up carried out was the same as that described in the comparative experiment. The IV of the resultant polymer was 58.3 ml/g.

The polymer was then, as described in the comparative experiment, processed to give granulated materials and injection-molded to give plaques, the optical properties of which were measured.

Color result: dL*=3.58, db*=−5.56

### Example 2 of the Invention

The synthesis used the same freshly distilled NMP from the comparative experiment, but 0.15 g of a 2 molar aqueous NaOH solution had also been added to each liter of this NMP. The molar NaOH/NMS ratio was 0.5.

The polymer synthesis and polymer work-up carried out was the same as that described in the comparative experiment. The IV of the resultant polymer was 58.3 ml/g.

The polymer was then, as described in the comparative experiment and example 1, processed to give granulated materials and injection-molded to give plaques, the optical properties of which were measured.

Color result: dL*=2.21, db*=−3.32

The examples therefore provide evidence that the process of the invention achieves markedly better color results.

1-14. (canceled)

15. A process for the purification of a crude solvent stream comprising an N-alkyl-pyrrolidone and, as contaminant alongside this, the corresponding N-alkylsuccinimide, to give a pure solvent stream which meets the requirements for use in a process for the production of polymers, where compounds of higher and lower boiling point than the N-alkylpyrrolidone are removed by distillation, which comprises, prior to, during, or after the distillative purification, adding a hydroxide of an alkali metal or of an alkaline earth metal in a molar amount corresponding to a molar ratio of at least 0.1:1 for this hydroxide with respect to the respective N-alkylsuccinimide in the appropriate solvent stream present prior to, during, or after the distillative purification.

16. The process according to claim 15, wherein the process for the production of polymers is a process for the production of one or more polyarylene ether sulfones.

17. The process according to claim 15, wherein the process for the production of polymers is a polycondensation of aromatic bishalogen compounds and aromatic bisphenols or salts thereof in the presence of at least one alkali metal carbonate or ammonium carbonate.

18. The process according to claim 15, wherein the hydroxide of an alkali metal or of an alkaline earth metal is added in a molar amount corresponding to a molar ratio of from 0.1:1 to 10:1 with respect to the respective N-alkylsuccinimide in the appropriate solvent stream present prior to, during, or after the distillative purification.

19. The process according to claim 18, wherein the hydroxide of an alkali metal or of an alkaline earth metal is added in a molar amount corresponding to a molar ratio of from 0.2:1 to 2:1 with respect to the respective N-alkylsuccinimide in the appropriate solvent stream present prior to, during, or after the distillative purification.

20. The process according to claim 15, wherein the N-alkylpyrrolidone is M-ethylpyrrolidone or N-methylpyrrolidone.

21. The process according to claim 17, wherein the crude solvent stream is a recycling steam from a plant for the production of polyarylene ether sulfones via polycondensation of aromatic bishalogen compounds and of aromatic bisphenols or salts thereof in the presence of at least one alkali metal carbonate or ammonium carbonate or alkali metal hydrogencarbonate or ammonium hydrogencarbonate in N-alkylpyrrolidone as solvent, comprising
from 60 to 90% by weight of water,
from 10 to 40% by weight of N-alkylpyrrolidone, and
as contaminant detrimental to specification, up to 5000 ppm by weight of N-methyl-succinimide and, alongside this, up to 1000 ppm by weight of other substances with higher boiling point than N-alkylpyrrolidone, based in each case on the total weight of the recycling stream, where the entirety of the components does not exceed 100% by weight,
to give a pure N-alkylpyrrolidone steam which can be returned to the plant for the production of polyarylene ether sulfones.

22. The process according to claim 21, wherein the purification is carried out via final distillation in a final column, upstream of which there is a preliminary purification via evaporation in one or more evaporator stages to reduce the content of other substances with higher boiling point than N-methylpyrrolidone, where the final column receives one or more feed streams, and where the bottom stream from the final evaporator stage is discharged, and the bottom steam from the final column is entirely recycled into the final evaporator stage.

23. The process according to claim 22, wherein the preliminary purification via evaporation is carried out in two or three evaporator stages.

24. The process according to claim 23, wherein the addition of the hydroxide of the alkali metal or of the alkaline earth metal takes place in the first evaporator stage, where the hydroxide of the alkali metal or of the alkaline earth metal is introduced together with the recycling stream or separately therefrom into the first evaporator stage,

25. The process according to claim 23, wherein the addition of the hydroxide of the alkali metal or of the alkaline earth metal takes place in the second evaporator stage.

26. The process according to claim 23, wherein the addition of the hydroxide of the alkali metal or of the alkaline earth metal takes place in the third evaporator stage.

27. The process according to claim 15, wherein the hydroxide of the alkali metal or of the alkaline earth metal is sodium hydroxide.

28. The process according to claim 15, wherein the hydroxide of the alkali metal or of the alkaline earth metal is potassium hydroxide.

29. The process according to claim 20, wherein the N-alkylpyrrolidone is N-methylpyrrolidone.

30. The process according to claim 23, wherein the preliminary purification via evaporation is carried out in three evaporator stages.
